(19) United States
(12) Patent Application Publication
(10) Pub. No.: US 2026/0137587 A1
(43) Pub. Date: May 21, 2026

(54) VIBRATORY STIMULATION APPARATUS AND VIBRATORY STIMULATION METHOD

(71) Applicant: TOYOTA BOSHOKU KABUSHIKI KAISHA, Aichi (JP)

(72) Inventor: Tatsuo KAWAI, Chiryu-shi (JP)

(73) Assignee: TOYOTA BOSHOKU KABUSHIKI KAISHA, Aichi (JP)

(21) Appl. No.: 19/357,676

(22) Filed: Oct. 14, 2025

(30) Foreign Application Priority Data
Oct. 17, 2024 (JP) 2024-181923

(51) Int. Cl. A61H 23/02; A61N 1/04

(52) U.S. Cl. CPC A61H 23/02; A61N 1/0452; A61H 2201/0149; A61H 2201/0165; A61H 2201/10; A61H 2201/1695; A61H 2205/06; A61H 2205/106; A61H 2205/108

(57) ABSTRACT

A vibratory stimulation apparatus is provided that is configured to apply vibration to a body of a user. The vibratory stimulation apparatus includes a vibration applicator configured to increase an intramuscular pressure in a muscle in a part of the body relative to an intramuscular pressure in the muscle in a normal condition, and mechanically vibrate the muscle while the intramuscular pressure is in an increased state.

### CROSS-REFERENCE TO RELATED APPLICATIONS

This application claims the benefit of Japanese Patent Application No. 2024-181923 filed on Oct. 17, 2024 with the Japan Patent Office, and the entire disclosure of Japanese Patent Application No. 2024-181923 is incorporated herein by reference.

### BACKGROUND

The present disclosure relates to vibratory stimulation apparatus and vibratory stimulation method for promoting a user's venous return (also referred to as “milking action”) and neural activity (projection from individual receptors in muscles and tendons to motor neurons via sensory neurons and interneurons).

For example, Japanese Unexamined Patent Application Publication No. 2000-262578discloses a massager in which a vibration generator thereof is operated in a manner that the maximum amplitude of vibration (hereinafter, simply referred to as “amplitude”) generated by the vibration generator is kept at a fixed value.

### SUMMARY

To achieve sufficient venous return effect and neural activity effect, it is desirable to vibrate muscles with a relatively large amplitude. However, vibrating muscles with a large amplitude increases a load on a vibration generator, such as a vibration applicator. The present disclosure discloses an example of a vibratory stimulation apparatus and a vibratory stimulation method in view of this point.

A vibratory stimulation apparatus configured to apply vibration to a body of a user desirably includes, for example, a vibration applicator configured to increase an intramuscular pressure in a muscle in a part of the body relative to an intramuscular pressure in the muscle in a normal condition, and mechanically vibrate the muscle while the intramuscular pressure is in an increased state.

With this vibratory stimulation apparatus, the state of the muscle when vibrated becomes similar to a state in which the muscle is vibrated with a large amplitude. Thus, a load on the vibration applicator that generates vibration can be small.

The vibratory stimulation apparatus applies vibration to a muscle in a part of the body while the intramuscular pressure of the muscle is in the increased state relative to that in the normal condition. This works equivalent to applying further vibration to the muscle that has been subjected to vibration with an amplitude corresponding to the amount of the increase in the intramuscular pressure (hereinafter referred to as “additional amount”).

Thus, compared with the state of the muscle in the normal condition, the muscle is subjected to vibration with an amplitude obtained by adding the additional amount to an amplitude generated by mechanical vibration. This enhances physical stimulus, which acts on individual receptors in muscles and tendons, produced by actual mechanical vibration with a small amplitude. Consequently, the load on the vibration applicator that generates vibration can be small.

The vibratory stimulation apparatus may be configured as follows, for example.

The vibration applicator desirably includes: an auxiliary component including a protruding portion extending toward a specified user position; and an actuator configured to vibrate the auxiliary component.

The auxiliary component is desirably configured such that a surface pressure generated at a contact portion in a non-operating state of the actuator is smaller than a surface pressure at the contact portion generated in an operating state of the actuator. The contact portion is a portion of the protruding portion configured to contact the user. Thus, the vibratory stimulation apparatus can inhibit a situation in which the user has a feeling of awkwardness or discomfort in the non-operating state of the actuator.

It is desirable that at least the protruding portion includes a polymer material with a viscoelasticity. Thus, the vibratory stimulation apparatus can inhibit a situation in which the user has a feeling of awkwardness or discomfort in the non-operating state of the actuator with a simple configuration. In addition, it is possible, in the operating state of the actuator, to apply vibration to the user's muscle while reliably pressing the user's muscle.

It is desirable that the protruding portion is arranged in a recessed portion in a manner capable of emerging from and retracting into the recessed portion, and that the protruding portion is configured to be housed inside the recessed portion during the non-operating state of the actuator, and configured to be in a state protruding from the recessed portion in response to the operating state of the actuator.

Thus, a situation can be inhibited in which the user has a feeling of awkwardness or discomfort in the non-operating state of the actuator. In addition, it is possible, in the operating state of the actuator, to apply vibration to the user's muscle while reliably pressing the user's muscle.

The vibration applicator desirably includes: two electrodes arranged at a distance from each other and configured to apply an electric current to the muscles; and an actuator configured to vibrate a portion of the body of the user, the portion of the body being interposed between the two electrodes.

This makes it possible to increase the intramuscular pressure in a muscle in a part of the body relative to that in the normal condition without using the auxiliary component.

The electric current applied across the two electrodes is desirably at a current value so that muscle contraction generated by application of the electric current is smaller than a maximum voluntary contraction.

### BRIEF DESCRIPTION OF THE DRAWINGS

Some example embodiments of the present disclosure will be described hereinafter with reference to the accompanying drawings, in which:

FIG. 1 is a diagram illustrating a seat according to a first embodiment;

FIG. 2 is a block diagram of an electrical system of the seat according to the first embodiment;

FIG. 3 is a diagram illustrating an output waveform of a vibration applicator according to the first embodiment;

FIG. 4 is a diagram illustrating an auxiliary component according to the first embodiment;

FIGS. 5A and 5B are diagrams each illustrating an auxiliary component according to a second embodiment;

FIG. 6 is a diagram illustrating a vibration applicator according to a third embodiment;

FIG. 7 is a diagram illustrating a vibration applicator according to a fourth embodiment;

FIGS. 8A and 8B are diagrams each illustrating an auxiliary component according to a fifth embodiment;

FIG. 9 is a diagram illustrating a vibration applicator according to a sixth embodiment;

FIG. 10 is a diagram illustrating a seat according to a seventh embodiment; and

FIGS. 11A and 11B are diagrams each illustrating a vibratory stimulation apparatus according to an eighth embodiment.

### DETAILED DESCRIPTION OF EXEMPLARY EMBODIMENTS

Following embodiments represent examples of embodiments that fall within the technical scope of the present disclosure. In other words, matters and the like to specify the invention recited in the claims are not limited to any specific configuration, structure, or the like described in the following embodiments.

A member or portion that is described at least with a reference numeral is at least one in number except in a case of being accompanied by restrictive words such as “only one”. A vibratory stimulation apparatus according to the present disclosure comprises at least one of elements, such as a member or portion described at least with a reference numeral, and/or structural portions shown in the drawings.

### First Embodiment

The first embodiment is an example, in which a vibratory stimulation apparatus and a vibratory stimulation method according to the present disclosure are applied to a seat, such as a vehicle seat or an easy chair. The present embodiment is effective in preventing, especially, frailty (mental and physical deterioration due to aging).

### <1. Overview of Seat>

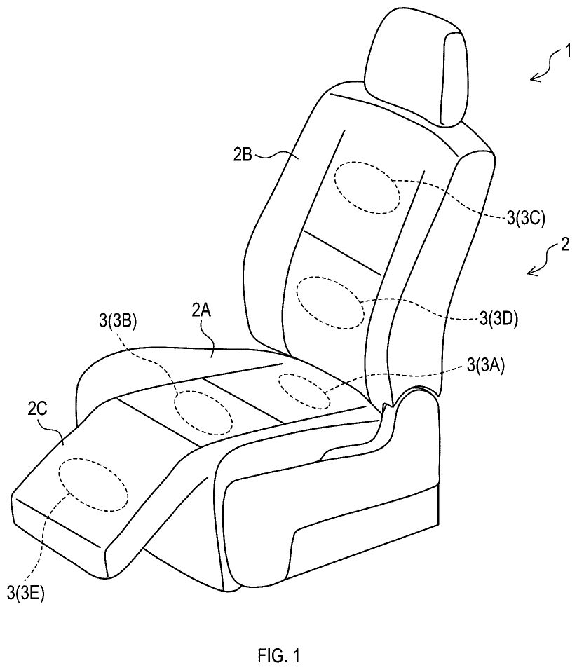

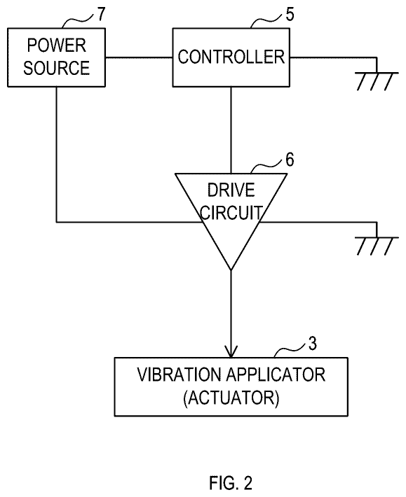

As shown in FIG. 1, a seat 1 according to the present embodiment includes a seat body 2, at least one vibration applicator 3 (two or more vibration applicators 3 in the present embodiment), and a controller 5 (see FIG. 2).

The seat body 2 includes at least one of a seat cushion 2A, a seatback 2B, or an ottoman 2C. The seat cushion 2A supports the lower body, such as legs, of a seated person. The seatback 2B supports the upper body, such as the waist and back, of the seated person. The ottoman 2C supports a lower part of the body below the knees of the seated person, such as the calves.

Each vibration applicator 3 applies vibration to the body of a user of the vibratory stimulation apparatus, that is, a user of the seat 1. In the present embodiment, at least one vibration applicator 3 is arranged in each of the seat cushion 2A, the seatback 2B, and the ottoman 2C.

Specifically, vibration applicators 3A and 3B are arranged in the seat cushion 2A. Vibration applicators 3C and 3D are arranged in the seatback 2B. A vibration applicator 3E is arranged in the ottoman 2C. Hereinafter, all or any of the vibration applicators 3A to 3E are referred to as “vibration applicator 3”. The vibration applicator 3 will be described in detail below.

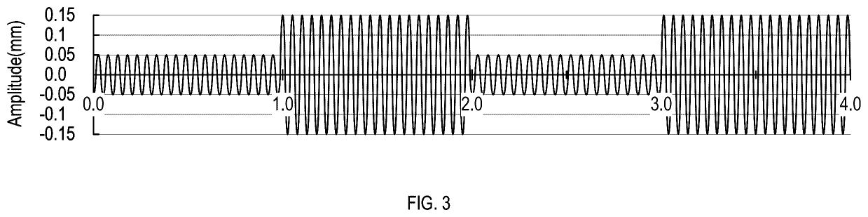

The controller 5 controls operation of the vibration applicator 3. The controller 5 can execute at least vibration mode control. The vibration mode control is, as shown in FIG. 3, to periodically vary the maximum amplitude of vibration applied by the vibration applicator 3 with the maximum amplitude kept at values greater than zero.

In FIG. 2, a drive circuit 6 drives the vibration applicator 3 (specifically, an actuator 32, which will be described below) in response to receiving a command from the controller 5. A power source 7 supplies an electric power to the controller 5 and the drive circuit 6, and the like.

### <2. Details of Vibration Applicator>

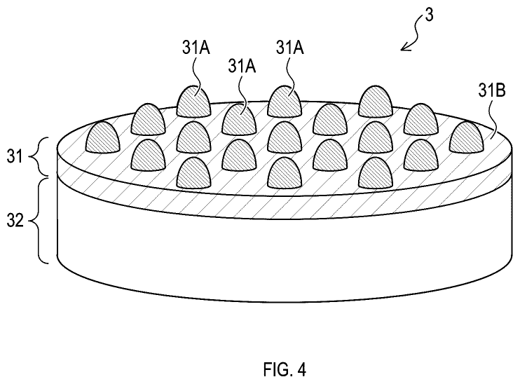

As shown in FIG. 4, the vibration applicator 3 includes an auxiliary component 31 and the actuator 32. The auxiliary component 31 includes at least one protruding portion 31A (two or more protruding portions 31A in the present embodiment) and a base-plate portion 31B.

Each protruding portion 31A protrudes toward the side where the user will be positioned (hereinafter referred to as “a specified user position”) (toward the upper side the drawing in FIG. 4). Each protruding portion 31A is integrated with the base-plate portion 31B having a plate shape. Each protruding portion 31A is configured in the form of a substantially spindle apparatus, having a cross-sectional area that decreases toward its tip.

Each protruding portion 31A is made of a viscoelastic polymer material. In other words, each protruding portion 31A is made of a polymer material that can achieve the following functions and effects regarding hardening and softening of the protruding portion 31A.

Each protruding portion 31A and the base-plate portion 31B are made of polyurethane or silicone rubber. The protruding portions 31A and the base-plate portion 31B are integrally formed into a single piece made of the polymer material.

The actuator 32 is a vibration generator that vibrates the auxiliary component 31. The actuator 32 includes a vibrating element of an electric actuator, such as an electromagnetic solenoid, an electrostatic actuator, or a piezoelectric device.

The base-plate portion 31B is joined to a vibrating portion of the actuator 32. Thus, when the actuator 32 is operated, the protruding portions 31A are vibrated via the base-plate portion 31B and displaced in the up-down direction of the drawing in FIG. 4.

### <3. Features of Vibratory Stimulation Apparatus of Present Embodiment>

Since each protruding portion 31A extends toward the specified user position, a part of the user's body, specifically the part which is located corresponding to the positions of the tips of the respective protruding portions 31A, is locally pressed when the user is seated on the seat 1.

Thus, when the actuator 32 is operated while the part of the body is locally pressed, the intramuscular pressure in some muscles is increased relative to that in a normal condition, and the muscles are mechanically vibrated while the intramuscular pressure is in an increased state. The term “normal condition” refers to a condition in which, for example, muscles are relaxed, in other words the user is relaxed and the muscles are not overly tense (contracted).

With this vibratory stimulation apparatus, the state of the muscles when vibrated becomes similar to a state in which the muscles are vibrated with a large amplitude. Thus, a load on the vibration applicator 3, that is the actuator 32, that generates vibration can be small.

The vibratory stimulation apparatus applies vibration to a muscle in a part of the body while the intramuscular pressure of the muscle is in the increased state relative to that in the normal condition. This works equivalent to applying further vibration to the muscle that has been subjected to vibration with an amplitude corresponding to the amount of the increase in the intramuscular pressure (hereinafter referred to as “additional amount”).

Thus, compared with the state of the muscles in the normal condition, the muscles are subjected to vibration with an amplitude obtained by adding the additional amount to an amplitude generated by mechanical vibration. This enhances physical stimulus, which acts on individual receptors in muscles and tendons, produced by actual mechanical vibration with a small amplitude. As a result, the load on the actuator 32 that generates vibration can be small.

Each protruding portion 31A is made of the aforementioned viscoelastic polymer material. This allows the auxiliary component 31 to work in a manner that a surface pressure generated at a contact portion of each protruding portion 31A configured to contact the user (hereinafter, simply referred to as “surface pressure”) in a non-operating state of the actuator 32 is smaller than the surface pressure generated in an operating state of the actuator 32.

Specifically, each protruding portion 31A when vibrating under a load is hardened due to strengthening of molecular bonds and an increase in internal friction. Each protruding portion 31A when not vibrating is softened due to relaxation of the molecular bonds and a decrease in the internal friction.

This means that each protruding portion 31A when not vibrating becomes softer than during vibration, and deforms to a have a large contact area with the user. Thus, the surface pressure in when the actuator 32 is stopped is smaller than the surface pressure in the operating state of the actuator 32.

When the user is seated while the actuator 32 is in the non-operating state, each protruding portion 31A easily deforms to disperse stress generated within the protruding portion, thereby reducing the pressure applied to the user. In the operating state of the actuator 32, each protruding portion 31A is stiffer due to viscoelasticity of the protruding portion 31A.

As a result, a situation can be inhibited in which the seated user has a feeling of awkwardness (discomfort) in the non-operating state of the actuator 32, that is when the vibratory stimulation apparatus is stopped. In the operating state of the actuator 32, that is when the vibratory stimulation apparatus is in operation, it is possible to apply vibration to the user's muscle while reliably pressing the user's muscle.

### Second Embodiment

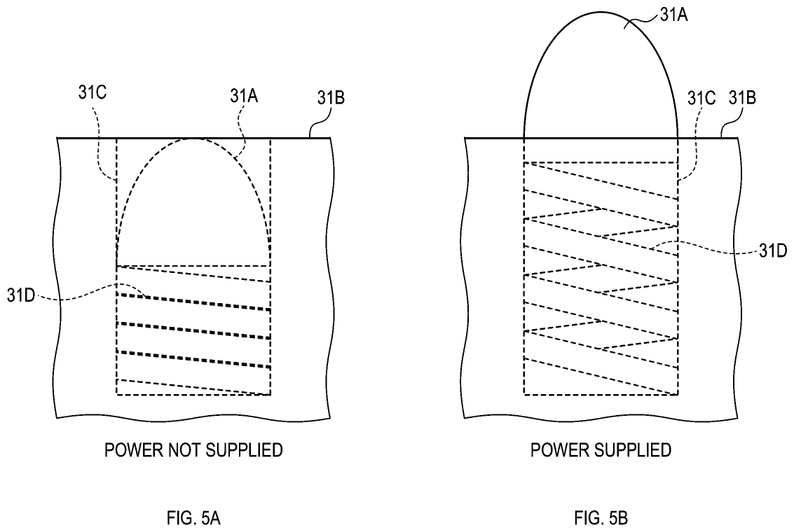

The individual protruding portions 31A of the aforementioned embodiment are integrally formed with the base-plate portion 31B into a single piece. The individual protruding portions 31A of the second embodiment are, as shown in FIGS. 5A and 5B, housed in recessed portions 31C of the base-plate portion 31B in a manner capable of emerging from and retracting into the respective recessed portions 31C.

The auxiliary component 31 includes (in the second embodiment, each recessed portions 31C is provided with) a displacement mechanism 31D configured to displace the protruding portion 31A. The displacement mechanism 31D causes the protruding portion 31A to be housed inside the recessed portion 31C when the actuator 32 is in the non-operating state (see FIG. 5A).

The displacement mechanism 31D causes the protruding portion 31A to be in a state protruding from the recessed portion 31C in the operating state of the actuator 32 (see FIG. 5B). The displacement mechanism 31D includes a coil made of a shape-memory alloy that changes shape depending on temperature.

Specifically, the controller 5 supplies electric power to the displacement mechanism 31D in the operating state of the actuator 32, and cuts off the electric power to the displacement mechanism 31D in the non-operating state of the actuator 32. This causes the protruding portion 31A to be housed inside the recessed portion 31C during the non-operating state of the actuator 32, and causes the protruding portion 31A to be in a state protruding from the recessed portion 31C when the actuator 32 is in the operating state.

The coil of the displacement mechanism 31D shown in FIGS. 5A and 5B is configured to extend when heated, in other words receiving the electric power, and contract when not receiving the electric power. However, the second embodiment is not limited to this configuration. For example, the displacement mechanism 31D may be configured to contract while receiving power supply and extend when not receiving power supply, and configured such that the protruding portion 31A in its extended state is housed inside the recessed portion 31C.

The displacement mechanism 31D is only required to be configured to cause the protruding portion 31A to be housed inside the recessed portion 31C during the non-operating state of the actuator 32, and cause the protruding portion 31A to be in a state protruding from the recessed portion 31C when the actuator 32 is in the operating state.

The same elements and so on as in the aforementioned embodiment are assigned with the same reference numerals as in the aforementioned embodiment. Thus, the same explanations are not repeated in the second embodiment. The protruding portion 31A according to the second embodiment may be made of any one of a viscoelastic material or a material, such as polyoxymethylene (POM), that can be regarded as a stiff body.

### Third Embodiment

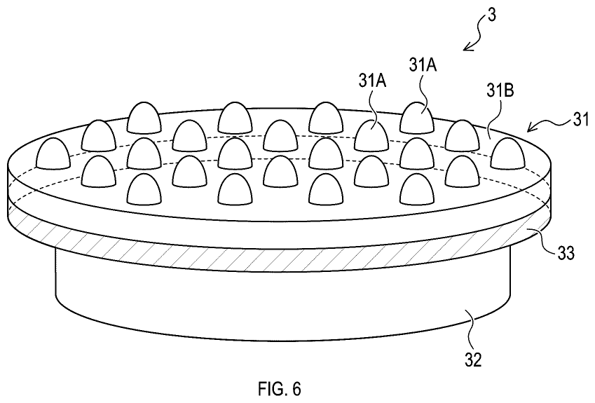

As shown in FIG. 6, the vibration applicator 3 according to the third embodiment includes a vibration plate 33 interposed between the auxiliary component 31 and the actuator 32. The vibration plate 33 reliably diffuses vibration of the actuator 32 to the protruding portions 31A.

The vibration plate 33 is a plate material made of metal or resin, such as POM, and is for use to vibrate the auxiliary component 31 larger than the vibrating portion of the actuator 32. The vibration plate 33 has a shape, such as a flat-plate shape, a shape having a curvature that follows the shape of the vibrating portion, and the like that should be selected as appropriate.

FIG. 6 illustrates the auxiliary component 31 according to the first embodiment. However, the third embodiment is not limited to this. For example, the auxiliary component 31 may be that of the second embodiment. The same elements and so on as in the aforementioned embodiments are assigned with the same reference numerals as in the aforementioned embodiments. Thus, the same explanations are not repeated in the third embodiment.

### Fourth Embodiment

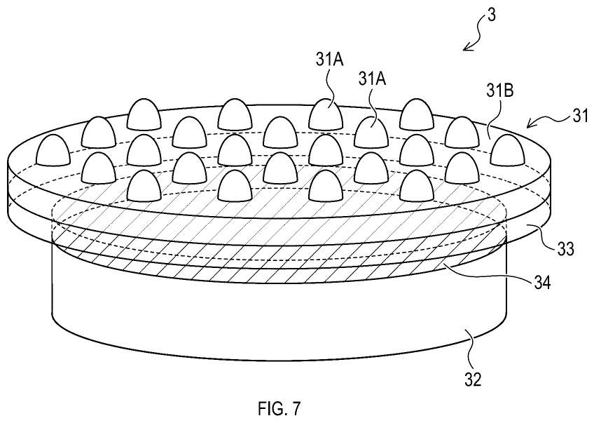

As shown in FIG. 7, the vibration applicator 3 according to the fourth embodiment includes a shock absorber 34 interposed between the auxiliary component 31 and the actuator 32. The shock absorber 34 includes a viscoelastic body (for example, polyurethane or silicone rubber) that absorbs and disperses vibration and shaking other than vehicle vibration and the like caused by the actuator 32.

FIG. 7 illustrates the auxiliary component 31 according to the third embodiment. However, the fourth embodiment is not limited to this. For example, the auxiliary component 31 may be that of the first or the second embodiment. The same elements and so on as in the aforementioned embodiments are assigned with the same reference numerals as in the aforementioned embodiments. Thus, the same explanations are not repeated in the fourth embodiment.

### Fifth Embodiment

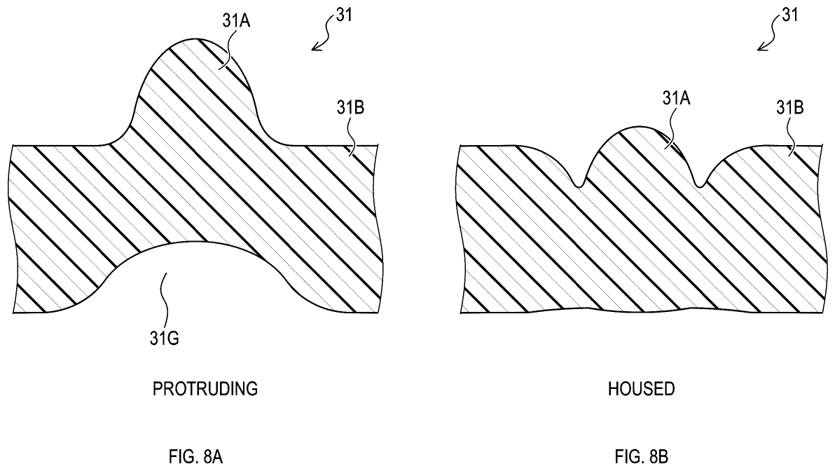

As shown in FIG. 8A, the auxiliary component 31 according to the fifth embodiment includes a recessed portion 31G formed on the opposite side of the base-plate portion 31B from the protruding portion 31A and in a portion corresponding to the position of the protruding portion 31A. The recessed portion 31G is recess toward the protruding portion 31A.

As in the first embodiment, the individual protruding portions 31A are integrally formed with the base-plate portion 31B in a single piece made of a viscoelastic polymer material (for example, polyurethane or silicone rubber).

Thus, the protruding portions 31A and the base-plate portion 31B when vibrated, harden as described above. As a result, the protruding portions 31A turn into a state protruding toward the specified user position (toward the upper side of the drawing in FIG. 8A) as shown in FIG. 8A.

In contrast, the protruding portions 31A and the base-plate portion 31B, when not vibrated, soften, and the stiffness of the protruding portions 31A adjacent to their roots is reduced. As a result, the protruding portions 31A pressed by the weight of the user turn into a state as if the protruding portions 31A are housed inside the base-plate portion 31B as shown in FIG. 8B.

Thus, also in the fifth embodiment, a situation can be inhibited in which the seated user has a feeling of awkwardness (discomfort) when the vibratory stimulation apparatus is stopped. In addition, it is possible when the vibratory stimulation apparatus is in operation to apply vibration to the user's muscle while reliably pressing the user's muscle.

The same elements and so on as in the aforementioned embodiments are assigned with the same reference numerals as in the aforementioned embodiments. Thus, the same explanations are not repeated in the fifth embodiment.

### Sixth Embodiment

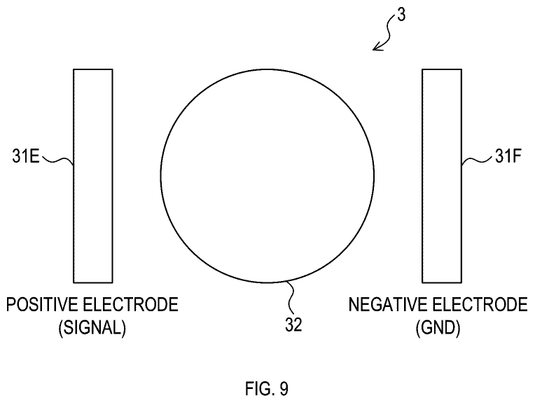

As shown in FIG. 9, the vibration applicator 3 according to the sixth embodiment includes two electrodes 31E and 31F and the actuator 32. The vibration applicator 3 does not have the auxiliary component 31 of the aforementioned embodiments.

The two electrodes 31E and 31F are arranged at a distance from each other and configured to apply an electric current to some of the muscles. The actuator 32 according to the sixth embodiment directly vibrates some of the muscles without the auxiliary component 31.

In other words, the actuator 32 according to the sixth embodiment vibrates the part of the user's body interposed between the two electrodes 31E and 31F (hereinafter referred to as “target area”). The controller 5 operates the actuator 32 while an electric current is applied across the two electrodes 31E and 31F.

When the actuator 32 is stopped, the application of the electric current across the electrodes 31E and 31F is stopped. The electric current applied across the electrodes 31E and 31F is at a current value so that muscle contraction generated by the application of the electric current is smaller than the maximum voluntary contraction.

As a result, vibration is applied to the target area while the muscles of the target area are contracted. For this reason, the vibration applicator 3 according to the sixth embodiment is also configured to increase the intramuscular pressure in the muscles relative to that in the normal condition and mechanically vibrate the muscles while the intramuscular pressure is in the increased state.

Thus, in the sixth embodiment, as in the aforementioned embodiments, the amplitude of vibration applied to the muscles is increased even if the amplitude of actual mechanical vibration is small. As a result, the load on the actuator 32 that generates vibration can be small.

In FIG. 9, the electrodes 31E and 31F are positioned off the actuator 32. However, the sixth embodiment is not limited to this. The electrodes 31E and 31F may be, for example, arranged on the vibrating portion of the actuator 32.

The same elements and so on as in the aforementioned embodiments are assigned with the same reference numerals as in the aforementioned embodiments. Thus, the same explanations are not repeated in the sixth embodiment.

### Seventh Embodiment

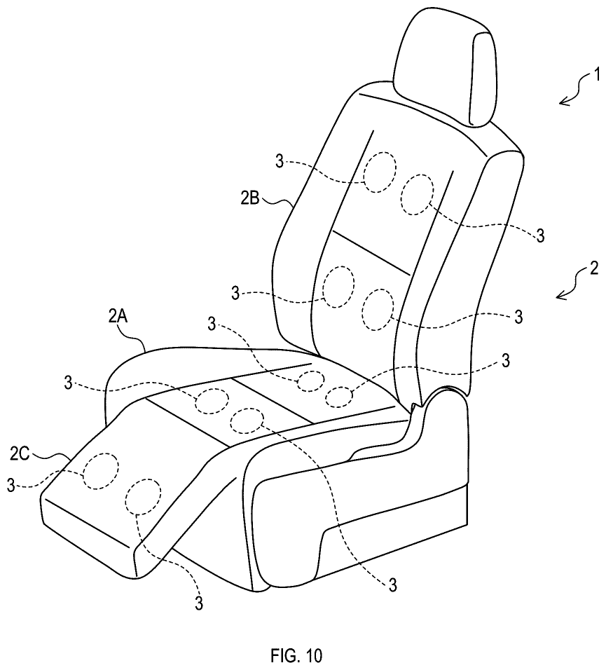

As shown in FIG. 10, two or more of the vibration applicator 3 are provided for each of the right half of the body and the left half of the body in the seventh embodiment. The controller 5 according to the seventh embodiment controls the operation of each vibration applicator 3 by, for example, shifting the period of amplitude variation by half a cycle between the vibration applicators 3 for the right half of the body and the vibration applicators 3 for the left half of the body.

The way to control the vibration applicators 3 with the controller 5 is not limited to the one described above. The same elements and so on as in the aforementioned embodiments are assigned with the same reference numerals as in the aforementioned embodiments. Thus, the same explanations are not repeated in the seventh embodiment.

### Eighth Embodiment

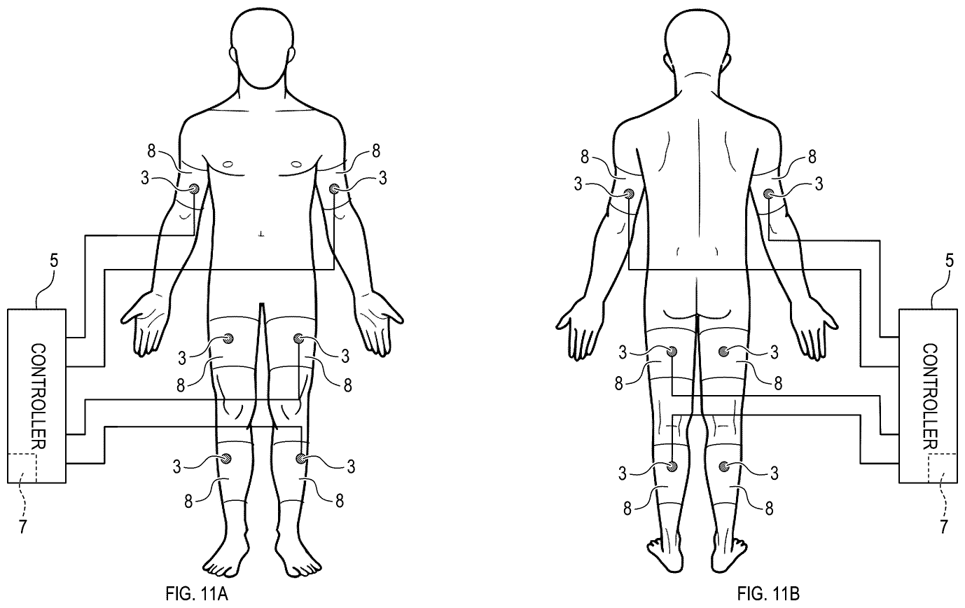

The aforementioned embodiments are examples in which a vibratory stimulation apparatus and a vibratory stimulation method according to the present disclosure are applied to a seat. As shown in FIG. 11A and FIG. 11B, the eighth embodiment provides a vibratory stimulation apparatus that includes a fastener 8 configured to closely secure the vibration applicator 3 to the user's body.

The fastener 8 according to the eighth embodiment is a fastener easily attachable and detachable, such as a hook-and-loop fastener. The attachment positions of the vibration applicators 3 shown in FIG. 11A and FIG. 11B are examples. The muscles to be vibrated may be any part of the body, such as a muscle belly or a tendon area.

The same elements and so on as in the aforementioned embodiments are assigned with the same reference numerals as in the aforementioned embodiments. Thus, the same explanations are not repeated in the eighth embodiment.

### Other Embodiments

In the aforementioned embodiments, at least one vibration applicator 3 is arranged in each of the seat cushion 2A, the seatback 2B, and the ottoman 2C. However, the present disclosure is not limited to this. For example, the vibration applicator 3 may be arranged only in the seat cushion 2A, the seatback 2B, or the ottoman 2C.

The auxiliary component 31 according to the aforementioned embodiments is configured such that the surface pressure generated at the contact portion between the protruding portion 31A and the user in the non-operating state of the actuator 32 is smaller than the surface pressure generated in the operating state of the actuator 32. However, the present disclosure is not limited to this.

The protruding portion 31A according to the first embodiment is made of a viscoelastic polymer material (specifically, polyurethane or silicone rubber). However, the present disclosure is not limited to this. For example, the protruding portion 31A may be made of a resin, such as POM, with high hardness.

In the aforementioned embodiments, the actuator 32 is configured to be controlled so that the amplitude is periodically varied. However, the present disclosure is not limited to this. For example, the actuator 32 may be configured to be controlled so that the amplitude is constant.

The shape of each protruding portion 31A is not limited to those illustrated in the drawings. That is, each protruding portion 31A is characterized by formation of unevenness, such as a mountain-like shape and a wave-like shape, and thus not limited by its shape.

In the aforementioned embodiments, the vehicle seat of the present disclosure is applied to a car. However, the application of the invention disclosed herein is not limited thereto. In other words, the present disclosure may be applied to a seat used in vehicles such as railroad vehicles, ships and boats, and aircrafts, and also to a stationary seat used in theaters and households, for example.

Furthermore, the present disclosure is only required to coincide with the gist of the disclosure described in the aforementioned embodiments and thus should not be limited to the aforementioned embodiments. Therefore, the present disclosure may include a configuration obtained by combining at least two of the aforementioned embodiments, or a configuration obtained by eliminating part of the elements in the drawings or the elements described with reference numerals in the aforementioned embodiments.

What is claimed is:

1. A vibratory stimulation apparatus configured to apply vibration to a body of a user, the apparatus comprising:
a vibration applicator configured to increase an intramuscular pressure in a muscle in a part of the body relative to an intramuscular pressure in the muscle in a normal condition, and mechanically vibrate the muscle while the intramuscular pressure is in an increased state.

2. The apparatus according to claim 1, wherein
the vibration applicator includes:
an auxiliary component including a protruding portion extending toward a specified user position; and
an actuator configured to vibrate the auxiliary component.

3. The apparatus according to claim 2, wherein the auxiliary component is configured such that a surface pressure generated at a contact portion in a non-operating state of the actuator is smaller than a surface pressure generated at the contact portion in an operating state of the actuator, the contact portion being a portion of the protruding portion configured to contact the user.

4. The apparatus according to claim 2, wherein at least the protruding portion includes a polymer material with a viscoelasticity.

5. The apparatus according to claim 3, wherein
the protruding portion is arranged in a recessed portion in a manner capable of emerging from and retracting into the recessed portion, and
the protruding portion is configured to be housed inside the recessed portion during the non-operating state of the actuator, and configured to be in a state protruding from the recessed portion in response to the operating state of the actuator.

6. The apparatus according to claim 1, wherein
the vibration applicator includes:
two electrodes arranged at a distance from each other and configured to apply an electric current to the muscle; and
an actuator configured to vibrate a portion of the body of the user, the portion of the body being interposed between the two electrodes.

7. The apparatus according to claim 6, wherein the electric current applied across the two electrodes is at a current value so that muscle contraction generated by application of the electric current is smaller than a maximum voluntary contraction.

8. A vibratory stimulation method for applying vibration to a body of a user, the method comprising:
increasing an intramuscular pressure in a muscle in a part of the body relative to an intramuscular pressure in the muscle in a normal condition; and
mechanically vibrating the muscle while the intramuscular pressure is in an increased state.
